(19) United States
(12) Patent Application Publication
(10) Pub. No.: US 2017/0052123 A1
(43) Pub. Date: Feb. 23, 2017

(54) SYSTEMS AND METHODS FOR EXTRACTION AND SURFACE-ENHANCED RAMAN SPECTROSCOPY DETECTION OF METAL NANOPARTICLES

(71) Applicants: Lili He, Amherst, MA (US); Baoshan Xing, Amherst, MA (US); Huiyuan Guo, Amherst, MA (US)

(72) Inventors: Lili He, Amherst, MA (US); Baoshan Xing, Amherst, MA (US); Huiyuan Guo, Amherst, MA (US)

(21) Appl. No.: 15/239,150

(22) Filed: Aug. 17, 2016

Related U.S. Application Data
(60) Provisional application No. 62/205,994, filed on Aug. 17, 2015.
(60) Provisional application No. 62/365,557, filed on Jul. 22, 2016.
(60) Provisional application No. 62/374,380, filed on Aug. 12, 2016.

(51) Int. Cl. G01N 21/77; G01N 33/20; G01N 21/65

(52) U.S. Cl. CPC G01N 21/77; G01N 21/658; G01N 33/20

(57) ABSTRACT

Systems and methods are provided herein for the extraction, detection and measurement of MNPs in complex matrices in particular, the method includes mixing a solution including a predetermined organic substance with a matrix including the metal nanoparticles, the predetermined organic substance adapted to bind to the metal nanoparticles, to facilitate extraction by an organic solvent and to provide a distinct SERS signal, the mixing resulting in a suspension, adding a solution of a predetermined organic extraction solvent to the suspension, extracting and separating the metal nanoparticles; the metal nanoparticles, after extraction, functionalized by binding with the predetermined organic substance, and extracting and separating the metal nanoparticles; the metal nanoparticles, after extraction, functionalized by binding with the predetermined organic substance; and performing SERS imaging on the functionalized metal nanoparticles in order to detect metals nanoparticles.

### CROSS REFERENCE TO RELATED APPLICATIONS

This application claims priority to and benefit of U.S. Provisional Application No. 62/205,994, entitled SYSTEMS AND METHODS FOR SURFACE-ENHANCED RAMAN SPECTROSCOPY DETECTION OF METAL NANOPARTICLES, filed on Aug. 17, 2015, U.S. Provisional Application No. 62/365,557, entitled SYSTEMS AND METHODS FOR SURFACE-ENHANCED RAMAN SPECTROSCOPY DETECTION OF METAL NANOPARTICLES, filed on Jul. 22, 2016, and U.S. Provisional Application No: 62/374,380, entitled METHODS OF USING FLAVONOID-ASSISTED MICROEXTRACTION AND SERS DETECTION FOR TITANIUM DIOXIDE (TiO2) NANOPARTICLES ANALYSIS, filed on Aug. 12, 2016, all of which are incorporated by reference herein in their entirety and for all purposes.

### STATEMENT REGARDING FEDERALLY SPONSORED RESEARCH OR DEVELOPMENT

This invention was made with government support under Grant No. NIFA 2015-67017-23070, awarded by the National Institute of Food and Agriculture of the U.S. Department of Agriculture. The federal government may have certain rights in the invention.

### BACKGROUND

The application of metal nanoparticles (collectively, MNPs) such as gold nanoparticles (AuNPs) and silver nanoparticles (AgNPs) in various sectors and consumer products continues to increase at a rapid pace. These applications can be grouped into three categories: food production/packaging, biomedicine and consumer goods. With respect to the food industry MNPs are largely used as coatings to prevent bacterial growth (due to antimicrobial properties). Specifically, applications include coatings on food preparation equipment, refrigerator storage containers and packaging materials. Similarly, with respect to the medical field, MNPs are frequently used as antimicrobial surface coatings for medical implants and other products such as of stents, breathing tubes, heart valves, catheters, surgical masks, and wound dressings. Therapeutic applications of MNPs have also been developed (e.g., use AuNPs in cancer treatment and drug delivery). Finally, with respect to consumer products, MNPs have wide spread usage in personal care products and cosmetics, textiles, electronics, household products, home improvement products, filtration/purification products and sanitization/cleaning products and the like. This widespread use is due not only to the antimicrobial characteristics of MNPs but also to enhanced thermal and electrical conductivity relative to non-nanoscale forms of the element.

The large scale application of MNPs increases the likelihood of human exposure and highlights the importance of thoroughly understanding nanoparticle fate and effects in biological systems. For example, previous studies have shown that AgNPs can enter human body through ingestion, inhalation and dermal contact, causing largely unknown risk and harm. Growing concerns over the potential risks of exposure to MNPs and the possible threats to environmental and biological systems have created a need for researchers to investigate the fate and behavior of MNPs. However, current analytical techniques cannot meet the requirements for probing MNPs in complex matrices (such as are characteristic of biological systems). Complex matrices may include, e.g., natural organic matter, metal cations, inorganic anions and other substances found in a given environment or biological system.

In published studies focused on consumer products (such as textiles and plush Toys) scanning electron microscopy (SEM), transmission electron microscopy (TEM), and energy dispersive X-ray spectroscopy (EDS) were used for particle identification and characterization of AgNPs. However, SEM and TEM can only provide size information in highly localized scanning areas and EDS is necessary for elemental identification. In addition, sample preparation and detection processes are labor intensive, somewhat destructive, and may alter the sample in unknown ways.

In terms of elemental quantification, graphite furnace atomic absorption spectrometry (GFAA), inductively coupled plasma optical emission spectrometer (ICP-OES) or inductively coupled plasma mass spectrometry (ICP-MS) can be used to determine Ag content. Although highly sensitive, spectrometric methods cannot differentiate AgNPs from silver ions or bulk particles and are less than ideal for providing spatial distribution information within the sample. Laser ablation ICP-MS can be used to gain spatial information on elemental distribution within the sample but still only provides total element content with no information on particle size or transition state. Single particle ICP-MS (sp-ICP-MS) can distinguish particle size but the resolution can be low; in addition, this technique requires assumptions about particle morphology and highly complex data analysis. Field flow fractionation ICP-MS (FFF-ICP-MS) has superb resolution but is expensive and labor intensive.

Conventional techniques for analyzing AgNPs are mainly facing 3 issues; complex sample preparation, matrix interference and speciation disability. Advanced techniques such as synchrotron X-ray absorption near-edge spectroscopy (XANES), single particle ICP-MS (sp-ICP-MS) and field flow fractionation ICP-MS (FFF-ICP-MS) are promising techniques that can address these issues. However, they may have other problems. For example, XANES analysis is not only complicated, but also access is usually limited due to the expensive and few available facilities. Single particle ICP-MS (sp-ICP-MS) is more available, but it requires monodisperse and spherical NPs with size >20 nm, FFF-ICP-MS is able to characterize AgNPs in different media. However, the method development is sophisticated and time-consuming due to the requirement of a large dataset for method optimization. The lack of methodology for effectively analyzing AgNPs in complex matrices has been a limiting factor for studying the environmental and biological fate and impacts of AgNPs.

A similar situation exists for other metal nanoparticles, such as titanium dioxide. In the past decades, titanium dioxide (TiO₂) is considered as an inert and safe material and has been used in many applications. Approximately 7 million tons of bulk TiO₂ are produced annually and used as white pigment in order to provide whiteness and opacity to products such as paints, coatings, plastics, papers, inks, foods, pills, as well as most toothpastes. More recently, with the development of nanotechnologies TiO₂ nanoparticles with numerous novel and useful properties have been manufactured and used, especially in personal care products such as topical sunscreens and cosmetic. By 2010, the production of TiO₂ nanoparticles had increased to 5000 tons and is expected to grow to 60,000 tons per year until 2025. Study also shows that there are approximately 36% TiO₂ nanoparticles in food-grade TiO₂ (E171) which are less than 100 nm in at least one dimension, and could readily disperse in water as fairly stable colloids. Consequentially, the large scale application of TiO₂ nanoparticles increases the likelihood of human exposure and entrance of this material into the environment. However, increasing studies showed that TiO₂ nanoparticles pose considerable risks to human health and environment. Mechanistic toxicological studies show that TiO₂ nanoparticles predominantly cause adverse effects via induction of oxidative stress resulting in cell damage, genotoxicity, inflammation, immune response etc. Recently, TiO₂ nanoparticles have been classified as “possible carcinogenic to humans” by the International Agency for Research on Cancer and as occupational carcinogen by the National Institute for Occupational Safety and Health, based on the experimental evidence from animal inhalation studies. Therefore, it is significantly important to assess the levels of TiO₂ nanoparticles in food, environmental samples and consumer products.

The conventional method for analyzing TiO₂ nanoparticles in complex matrices relies on the sample digestion with concentrated acid, followed by quantification with inductively coupled plasma mass spectrometry (ICP-MS) or inductively coupled plasma optical emission spectrometer (ICP-OES) for titanium element. Normally, hydrofluoric acid is used which is tremendously dangerous. Although highly sensitive, these method are still limited to many disadvantages: destruction of nanoparticles, the use of toxic reagents, the need of high level of expertise, complicating procedure and time-consuming. Extraction of TiO₂ nanoparticles from matrices using water or organic solvents is a promising way instead of digestion. Efficient hydrophobization and solvent microextraction method has been developed for determination of trace TiO₂ nanoparticles in natural water; however, the use of toxic organic solvents (methanol etc.) and the need of sample digestion for ICP-MS test hinder its practical applications, especially for on-site detection. Therefore, there are ever-increasing demands for developing a simple, fast and green method to analyze (extract and detect) TiO₂ nanoparticles in nondestructive manner.

Surface-enhanced Raman spectroscopy (SERS) is an emerging technique that combines Raman spectroscopy with nanotechnology. The analytical platform retains the inherent advantages of Raman spectroscopy, including small sample size, minimal sample preparation, rapid spectrum collection and characteristic fingerprint for specific analytes. However, SERS also overcomes two major drawbacks of Raman spectroscopy: low sensitivity and fluorescence interference. In conventional applications, nanoscale metals, such as silver and gold are common SERS substrates, and can enhance the sensitivity of Raman spectroscopy by as much as 10¹⁴ to 10¹⁵ fold. The enhancement mechanism lies in the electromagnetic field enhancement attributed to localized surface plasmon resonance (LSPR) as well as chemical enhancement due to charge transfer between the analyte and the substrate. With these enhancements, SERS is sensitive enough for detecting ultratrace analytes as low as pica-molar and femto-molar levels, and perhaps even single molecules. Slight alterations in molecular orientation and structure can also be resolved by SERS. In addition, low background auto-fluorescence can be achieved in SERS by selecting less energetic excitation or by detecting the analytes close to the SERS-active metal surface with a quenching effect. These characteristics make SERS a simple, rapid, nondestructive, reliable, and sensitive technique that is finding increasing use in Chemistry, molecular biology, medicine, food analysis, and environmental contaminant detection.

Conventional SERS-based investigations have been focusing on the use of Ag and/or Au nanosubstrates to detect metal-sorbed chemical or biological analytes. In this way, in conventional SERS systems and methods have utilized Ag and/or Au nanosubstrates as indicators for detecting other particles/molecules which form the analytical focus. Thus, SERS has not been applied for detecting MNPs as an analyte.

For at least the foregoing reasons there exists a need for the development of simple, rapid and accurate systems and methods for the extraction, detection and measurement of MNPs in complex matrices. Moreover, there exists a need for systems and methods for identifying and evaluating indicators for use in SERS-based detection of MNPs. These and other needs are met by way of the present disclosure.

### SUMMARY

Systems and methods are provided herein for the extraction, detection and measurement of MNPs in complex matrices.

In one or more embodiments, the method of these teachings for extraction and detection of metal nanoparticles from matrices includes mixing a solution including a predetermined organic substance with a matrix including the metal nanoparticles, the predetermined organic substance adapted to bind to the metal nanoparticles, to facilitate extraction by an organic solvent and to provide a distinct SERS signal, the mixing resulting in a suspension, adding a solution of a predetermined organic extraction solvent to the suspension, extracting and separating the metal nanoparticles; the metal nanoparticles, after extraction, functionalized by binding with the predetermined organic substance, and extracting and separating the metal nanoparticles; the metal nanoparticles, after extraction, functionalized by binding with the predetermined organic substance; and performing SERS imaging on the functionalized metal nanoparticles in order to detect metals nanoparticles.

In one instance, the metal nanoparticles are silver nanoparticles; and the predetermined organic substance is a ligand adapted to bind on surfaces of the silver nanoparticles, adapted to facilitate extraction by the organic solvent and adapted to produce the distinct SERS signal.

In one exemplary embodiment, the ligand includes a thiol group for binding with the silver nanoparticles, a carbon-carbon chain at another end to facilitate extraction, and a Raman active group.

In one or more embodiments, the composition of these teachings for extraction and detection of metal nanoparticles from matrices includes a solution including a predetermined organic substance; the predetermined organic substance adapted to bind to the metal nanoparticles, adapted to facilitate extraction by an organic solvent and adapted to provide a distinct SERS signal; the solution including the predetermined organic substance being mixed with a matrix including the metal nanoparticles; mixing resulting in a suspension; and a solution of a predetermined organic extraction solvent, the solution of the predetermined organic extraction solvent being added to the suspension, wherein the metal nanoparticles can be extracted and separated and detected by SERS imaging.

In one instance, the metal nanoparticles are silver nanoparticles; and the predetermined organic substance is a ligand adapted to bind on surfaces of the silver nanoparticles, adapted to facilitate extraction by the organic solvent and adapted to produce the distinct SERS signal.

In one exemplary embodiment, the ligand includes a thiol group for binding with the silver nanoparticles, a carbon-carbon chain at another end to facilitate extraction, and a Raman active group.

Other embodiments are also disclosed.

### BRIEF DESCRIPTION OF THE DRAWINGS

For a better understanding of the present teachings, together with other and further needs thereof, reference is made to the accompanying drawings and detailed description and its scope will be pointed out in the appended claims.

FIG. 1 depicts an embodiment of the method of these teachings for extracting and detecting silver nanoparticles, according to the present disclosure;

FIG. 2 depicts a graphical description of the embodiment of the method of these teachings for extracting and detecting silver nanoparticles.

FIG. 3A is a pictorial representation of separation before extraction, for different transfer agents and different concentrations of one transfer agent, in the embodiment of the method of these teachings for extracting and detecting silver nanoparticles;

FIG. 3B shows a graphical representation of the UB-visible spectra of silver nanoparticles in aqueous phase after extraction according to one embodiment these teachings for different concentrations of one transfer agent;

FIG. 4 depicts SERS spectra of different concentrations of silver nanoparticles in the interlayer after extraction according to one embodiment these teachings for different concentrations of one transfer agent;

FIG. 5 depicts SERS spectra of different concentrations of silver nanoparticles in the interlayer and in the organic phase after extraction according to one embodiment these teachings;

FIG. 6A shows SERS spectra of different concentrations of silver nanoparticles after extraction according to one embodiment these teachings;

FIG. 6B shows Raman intensity versus concentration of silver nanoparticles, after extraction according to one embodiment these teachings;

FIG. 7 shows SERS spectra of different concentrations of silver nanoparticles in wheat leaves and in the absence of wheat leaves, after extraction according to one embodiment these teachings;

FIG. 8A shows a schematic illustration of flavonoid-assisted extraction method for TiO₂ nanoparticles, according to the present disclosure;

FIG. 8B are photographs of flavonoid-based phase separation, according to the present disclosure; selected flavonoid is myricetin (MYC); and

FIG. 8C is a graphical representation of SERS spectra of MYC-adsorbed TiO₂ nanoparticles from the interlayer (NP=nanoparticle. NPs=nanoparticles), according to the present disclosure.

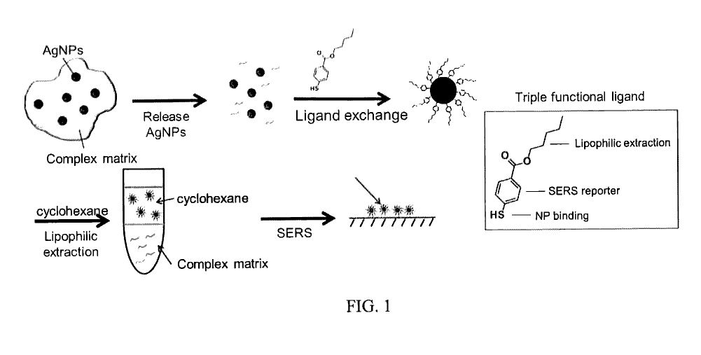

### DETAILED DESCRIPTION

The following detailed description presents the currently contemplated modes of carrying out the invention. The description is not to be taken in a limiting sense, but is made merely for the purpose of illustrating the general principles of the invention, since the scope of the invention is best defined by the appended claims.

Systems and methods are provided herein for the extraction, detection and measurement of MNPs in complex matrices.

In one or more embodiments, the method of these teachings for extraction and detection of metal nanoparticles from matrices includes mixing a solution including a predetermined organic substance with a matrix including the metal nanoparticles, the predetermined organic substance adapted to bind to the metal nanoparticles, to facilitate extraction by an organic solvent and to provide a distinct SERS signal, the mixing resulting in a suspension, adding a solution of a predetermined organic extraction solvent to the suspension, extracting and separating the metal nanoparticles; the metal nanoparticles, after extraction, functionalized by binding with the predetermined organic substance, and extracting and separating the metal nanoparticles; the metal nanoparticles, after extraction, functionatized by binding with the predetermined organic substance; and performing SERS imaging on the functionalized metal nanoparticles in order to detect metals nanoparticles.

In one or more embodiments, the composition of these teachings for extraction and detection of metal nanoparticles from matrices includes a solution including a predetermined organic substance; the predetermined organic substance adapted to bind to the metal nanoparticles, adapted to facilitate extraction by an organic solvent and adapted to provide a distinct SERS signal; the solution including the predetermined organic substance being mixed with a matrix including the metal nanoparticles; mixing resulting in a suspension; and a solution of a predetermined organic extraction solvent, the solution of the predetermined organic extraction solvent being added to the suspension, wherein the metal nanoparticles can be extracted and separated and detected by SERS imaging.

Exemplary embodiments are presented below. It should be noted that these teachings are not limited only to the exemplary embodiments.

In one instance, the metal nanoparticles are silver nanoparticles; and the predetermined organic substance is a ligand adapted to bind on surfaces of the silver nanoparticles, adapted to facilitate extraction by the organic solvent and adapted to produce the distinct SERS signal.

In one exemplary embodiment, the ligand includes a thiol group for binding with the silver nanoparticles, a carbon-carbon chain at another end to facilitate extraction, and a Raman active group.

In one instance, the solution including the ligand is 4-mercaptobenzoic acid (4-MBA) dissolved in methanol. In one instance, the predetermined organic extraction solvent includes cyclohexane having a predetermined transfer agent in solution. In one embodiment, the predetermined transfer agent is at least one of octadecylamine (ODA) and tetraoctylammoniumbromide (TOAB). Embodiments in which the predetermined transfer agent is TOAB and in which a molar concentration of the TOAB is between 200 and 500 μmol per liter (μM), preferably a molar concentration of the TOAB being about 250 μmol per liter (μM), are disclosed hereinbelow.

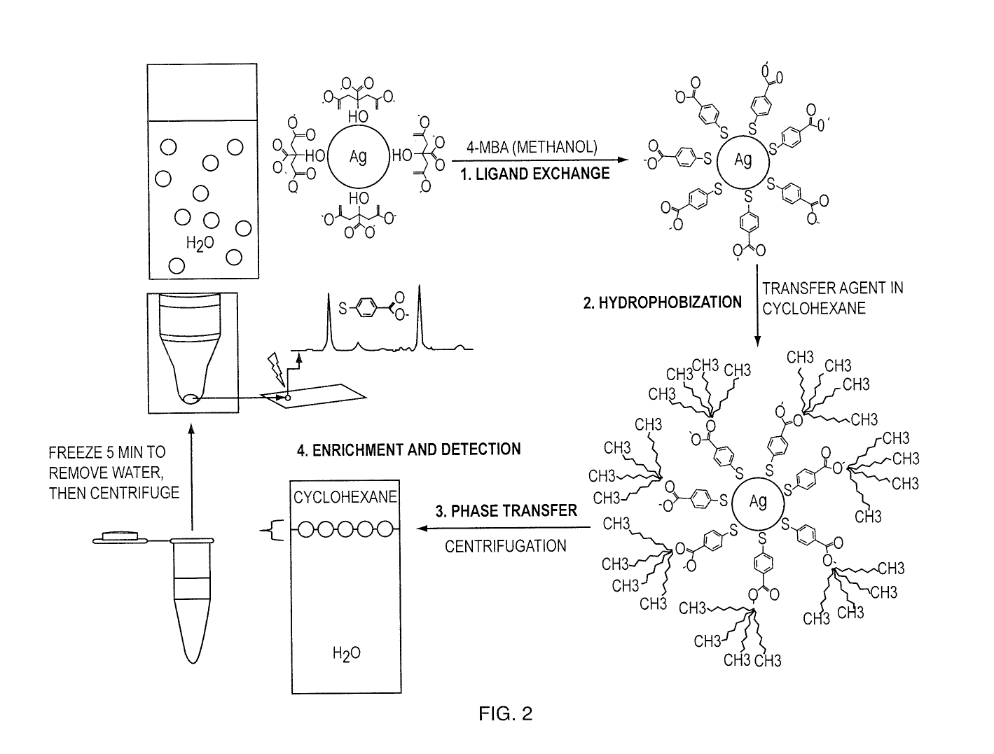

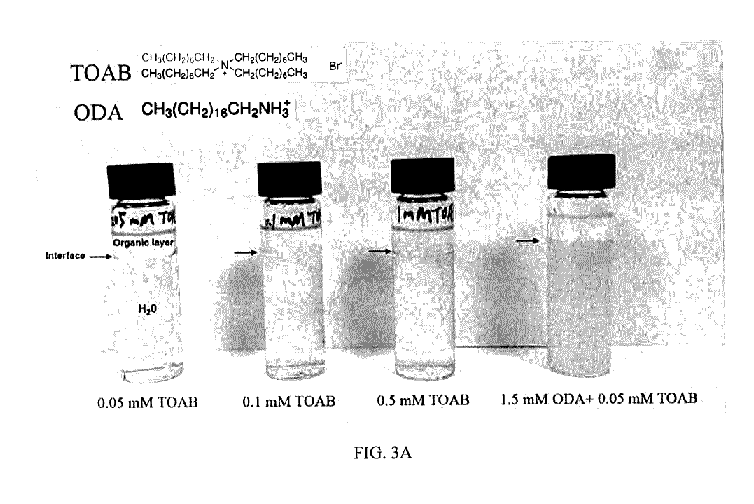

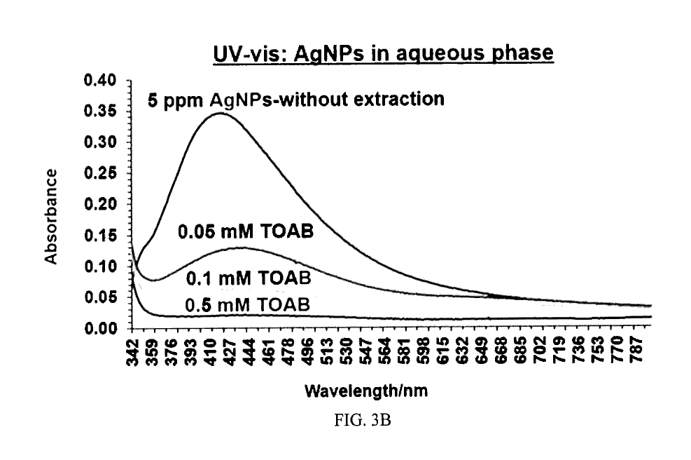

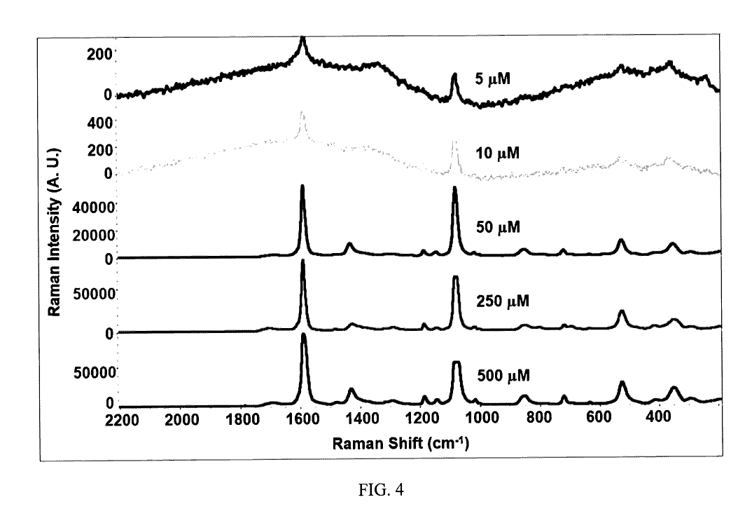

The use of 4-mercaptobenzoic acid (4-MBA) and tetraoctylammoniumbromide (TOAB) to form the triple functional ligands is disclosed hereinbelow. As shown in FIG. 2, 4-MBA (dissolved in methanol) was mixed with aqueous solutions of citrate-AgNPs at a volume ratio of 1:10. In this study, 4-MBA plays three roles. The first one is to surface modify citrate-coated AgNPs through replacing citrate with thiol group. The second is to form acid-base pairs with transfer agent (surfactant) through electrostatic attraction, which will increase the hydrophobicity of AgNPs. The last but not the least, 4-MBA, with distinct SERS peaks at around 1100 and 1590 cm⁻¹, can act as an AgNP probe. To facilitate the adsorption of 4-MBA to AgNPs, the mixture was ultrasonicated for 3 min and further shaken for 0-2 h at 150 rpm on a platform shaker (Innova 2100, Eppendorf). To extract the 4-MBA modified AgNPs, 0.25-1 mL cyclohexane containing transfer agent was added into the suspension. Potential phase transfer agents such as octadecylamine (ODA) and tetraoctylammoniumbromide (TOAB), were used to enhance the hydrophobicity of AgNPs and further partition into water-cyclohexane interlayer or cyclohexane phase. After incubation for 0-60 min on the shaker, the mixture was centrifuged at 3 000 rpm for 5-10 min. Phase separation was achieved with cyclohexane on the top layer. As seen in FIG. 3A, the color disappearance in water phase indicates the extraction effectiveness. A previous study has reported that a combination of ODA and TOAB could extract AgNPs more efficiently than ODA alone (see Majedi, S. M.; Kelly, B. C.; Lee, H. K. Efficient hydrophobization and solvent microextraction for determination of trace nano-sized silver and titanium dioxide in natural waters. Anal. Chim. Acta 2013, 789, 47-57, which is incorporated by reference herein in its entirety and for all purposes). The present disclosure shows that compared with a combination of ODA and TOAB, TOAB alone showed better extraction ability. As a result, TOAB was used as the transfer agent in exemplary embodiment. Furthermore, the present disclosure shows that the extraction effectiveness increases with TOAB concentration increasing from 0.05 mM to 0.5 nM according to the decrease of UV-vis absorbance of aqueous AgNPs (FIG. 3B). SERS spectra displayed, as shown in FIG. 4, that the signal intensity increased with TOAB concentration increasing from 5 μM to 250 μM, and reached plateau afterwards, which are consistent with UV-vis data. Therefore, 250 μM TOAB was preferably used.

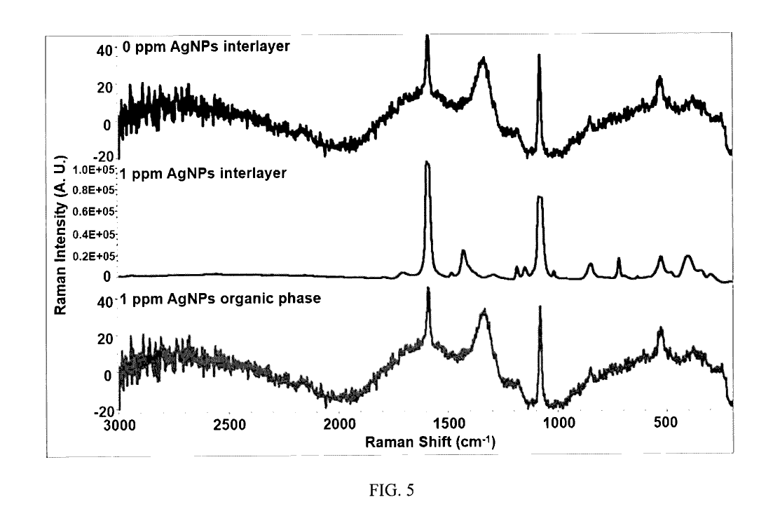

After phase separation, whether the AgNPs were extracted into the cyclohexane phase or in the interface determined how we sampled the AgNPs for SERS analysis. Both the interlayer solution and organic phase were detected. As we can see in FIG. 5, the interlayer had much higher signal intensity than the organic layer, which indicates that the extracted AgNPs were mainly concentrated in the interlayer. It has been previously reported that citrate coated on gold nanoparticles (AuNPs) can only be partially replaced in citrate-thiol ligand exchange due to a stabilized network formed through hydrogen bonding between carboxyl moieties (see Kalimuthu, P.; John, S. A. Studies on ligand exchange reaction of functionalized mercaptothiadiazole compounds onto citrate capped gold nanoparticles. Mater, Chem. Phys. 2010, 122 (2-3), 380-385, which is incorporated by reference herein in its entirety and for all purposes). Partial modification of AgNPs surface by 4-MBA and further formation of acid-base pairs between 4-MBA and TOAB lead to amphiphilic AgNPs, which explains why they were extracted in the interface.

Next, the organic phase and 0.5 mL top layer of water phase were collected. To remove the water phase, the mixture solution was placed at −20° C. with the tubes upside down for 7 min. Due to the freezing point difference between water (0° C.) and cyclohexane (6.55° C.), the cyclohexane layer was frozen while the water phase was still in liquid, which can be decanted easily, as shown in FIG. 2, The remaining solution was centrifuged at 10 000-13 300 rpm for 5 min to concentrate the amphiphilic AgNPs to a drop of water at the bottom of the tube, which was then placed onto a clean surface of a gold slide and dried in a fume hood. After drying, the samples were immediately detected by a DXR Raman Spectro-microscope (Thermo Scientific, Madison, Wis.), which consisted of a 780-nm laser with an output power of 5 mW, a 20× confocal microscope objective with 1.9 μm spot diameter, as well as a 50 μm slit width for 2 s integration time. The detection process was monitored using the OMNIC™ software (version 9.1). Ten spectra from each sample were chosen and averaged to a final spectrum using TQ Analyst software (version 8.0, Thermo Scientific).

To extract AgNPs from biological tissues, wheat plants, which were grown for three weeks in greenhouse (25° C. with 16 h/8 h (light/dark) cycle, light intensity ˜750 μmol m⁻² s⁻¹) were used as a model, The leaves of the harvested wheat plants were separated and grounded under liquid nitrogen. AgNPs colloids were spiked with the homogenized wheat leaves. After incubation with 4-MBA, the mixture was centrifuged at low speed (3 000 rpm, 5 min) to remove bulky plant residues. The supernatant was used for further extraction and detection using the same methods as described above.

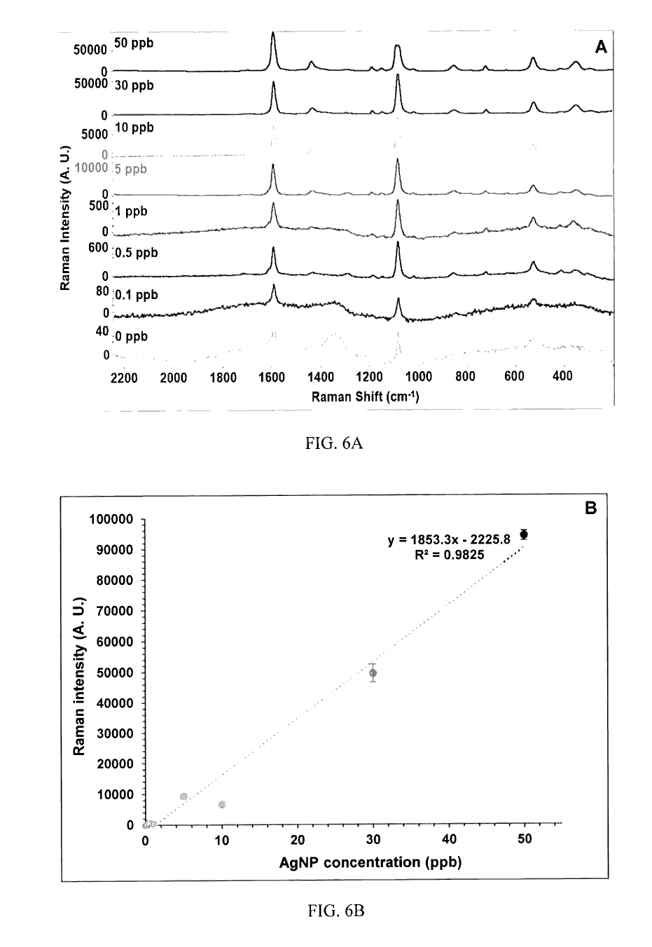

As shown in FIG. 6A, the hydrophohization-mediated extraction assisted SERS can detect AgNPs as low as 0.1 ppb, which is within the environmentally relevant levels of AgNPs²⁰. In addition, Raman intensity and AgNP concentration are linearly related (FIG. 6B), which shows the potential of the developed method to quantify AgNPs in real water systems.

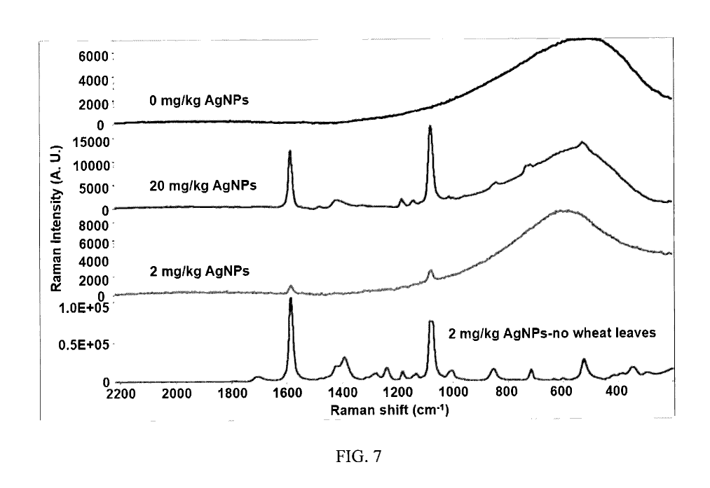

Using the developed method, AgNPs in wheat leaves. As shown in FIG. 7, compared with the blank control, the samples with AgNPs showed the enhanced Raman peaks of 4-MBA. Moreover, the signal intensity was dependent on AgNP concentration. 2 mg AgNPs/kg wheat leaves can be easily detected using the method of these teachings. In most in vivo studies employing mammalian species, the concentrations of AgNPs used were higher than 2 mg/kg. Therefore, the method of these teachings is well suited for in vivo studies to detect AgNPs in organisms.

In one instance, the metal nanoparticles are Titanium dioxide (TiO₂) nanoparticles; and the predetermined organic substance is a flavonoid that adapted to bind on surfaces of the silver nanoparticles, adapted to facilitate extraction by the organic solvent and adapted to produce the distinct SERS signal. In one instance, the predetermined organic extraction solvent was ethyl acetate, in one exemplary embodiment, the flavonoid used was myricetin (MYC).

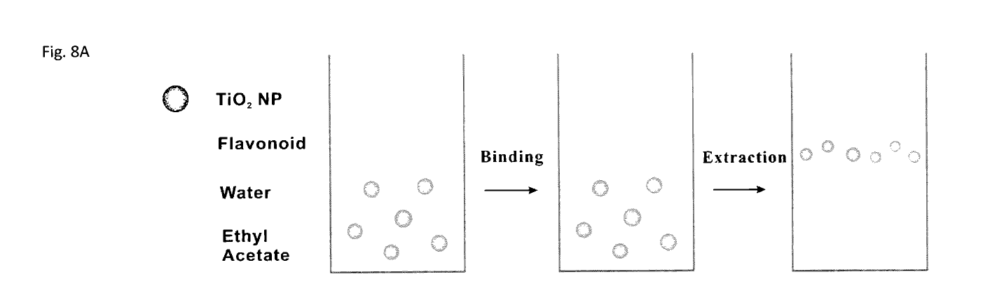

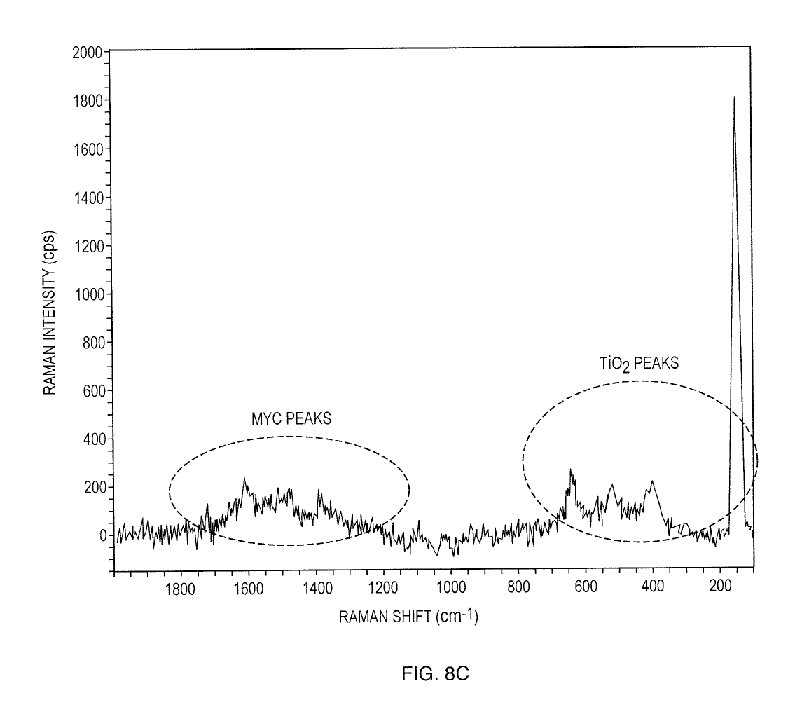

FIG. 8A schematically illustrates the design of flavonoid-assisted extraction method for TiO₂ nanoparticle from water. The TiO₂ nanoparticle dispersed in water are first mixed with flavonoid solution (flavonoid dissolved in ethanol) thoroughly and then incubated at room temperature for 2 hours to allow the binding between flavonoid and nanoparticle. Then, with the help of an organic extraction solvent (ethyl acetate) TiO₂ nanoparticles could be successfully extracted to form an interlayer between organic phase and aqueous phase. Finally, the extracted nanoparticles could be easily separated and detected by SERS. As shown in FIG. 8B, an interlayer containing TiO₂ nanoparticles was clearly observed between organic phase and aqueous phase when myricetin (MYC) was used as selected flavonoid. Control experiments were used to validate the performance of MYC-assisted nanoparticles extraction, In one instance, after vortexing 15 s to facilitate phase separation, the resulting interlayer containing flavonoid-adsorbed TiO₂ NPs between the organic and aqueous phase was separated and a portion was pipetted onto a gold slide and air dried for SERS measurement. The extracted TiO₂ nanoparticles were detected by SERS showing Raman peaks of both MYC and TiO₂ (FIG. 8C).

Although these teachings have been described with respect to various embodiments, it should be realized these teachings are also capable of a wide variety of further and other embodiments within the spirit and scope of the appended claims.

What is claimed is:

1. A method for extraction and detection of metal nanoparticles from matrices, the method comprising:
mixing a solution including a predetermined organic substance with a matrix including the metal nanoparticles; the predetermined organic substance adapted to bind to the metal nanoparticles, to facilitate extraction by an organic solvent and to provide a distinct SERS signal; the mixing resulting in a suspension;
adding a solution of a predetermined organic extraction solvent to the suspension;
extracting and separating the metal nanoparticles, the metal nanoparticles, after extraction, functionalized by binding with the predetermined organic substance; and
performing SERS imaging on the functionalized metal nanoparticles in order to detect metals nanoparticles.

2. The method of claim 1 wherein the metal nanoparticles are silver nanoparticles; and wherein the predetermined organic substance is a ligand adapted to bind on surfaces of the silver nanoparticles, adapted to facilitate extraction by the organic solvent and adapted to produce the distinct SERS signal.

3. The method of claim 2 wherein the ligand includes a thiol group for binding with the silver nanoparticles, a carbon-carbon chain at another end to facilitate extraction, and a Raman active group.

4. The method of claim 3 wherein the solution including the ligand is 4-mercaptobenzoic acid (4-MBA) dissolved in methanol.

5. The method of claim 4 wherein the predetermined organic extraction solvent comprises cyclohexane having a predetermined transfer agent in solution.

6. The method of claim 5 wherein the predetermined transfer agent is at least one of octadecylamine (ODA) and tetraoetylammoniumbromide (TOAB).

7. The method of claim 6 wherein the predetermined transfer agent is TOAB.

8. The method of claim 7 wherein a molar concentration of the TOAB is between 200 and 500 μmol per liter (μM).

9. A composition for extraction and detection of metal nanoparticles from matrices, the composition comprising:
a solution including a predetermined organic substance; the predetermined organic substance adapted to bind to the metal nanoparticles, adapted to facilitate extraction by an organic solvent and adapted to provide a distinct SERS signal; the solution including the predetermined organic substance being mixed with a matrix including the metal nanoparticles; mixing resulting in a suspension; and
a solution of a predetermined organic extraction solvent; the solution of the predetermined organic extraction solvent being added to the suspension;
wherein the metal nanoparticles can be extracted and separated and detected by SERS imaging.

10. The composition of claim 9 wherein the metal nanoparticles are silver nanoparticles; and wherein the predetermined organic substance is a ligand adapted to bind on surfaces of the silver nanoparticles, to facilitate extraction by the organic solvent and produce the distinct SERS signal.

11. The composition of claim 10 wherein the ligand includes a thiol group for binding with the silver nanoparticles, a carbon-carbon chain at another end to facilitate extraction, and a Raman active group.

12. The composition of claim 11 wherein the solution including the ligand is 4-mercaptobenzoic acid (4-MBA) dissolved in methanol.

13. The composition of claim 12 wherein the predetermined organic extraction solvent comprises cyclohexane having a predetermined transfer agent in solution.

14. The composition of claim 13 wherein the predetermined transfer agent is at least one of octadecylamine (ODA) and tetraoctylammoniumbromide (TOAB).

15. The composition of claim 14 wherein the predetermined transfer agent is TOAB.

16. The composition of claim 15 wherein a molar concentration of the TOAB is between 200 and 500 μmol per liter (μM).
